(19) United States
(12) Patent Application Publication
(10) Pub. No.: US 2025/0241375 A1
(43) Pub. Date: Jul. 31, 2025

(54) ATOMIZATION DEVICE

(71) Applicant: SHENZHEN WOODY VAPES TECHNOLOGY CO., LTD., Shenzhen (CN)

(72) Inventors: Yong YANG, Shenzhen (CN); Youming LIU, Shenzhen (CN); Wenlin HUANG, Shenzhen (CN)

(73) Assignee: SHENZHEN WOODY VAPES TECHNOLOGY CO., LTD., Shenzhen (CN)

(21) Appl. No.: 18/822,717

(22) Filed: Sep. 3, 2024

(30) Foreign Application Priority Data
Jan. 31, 2024 (CN) 202420250718.5
Jan. 31, 2024 (CN) 202420255548.X

(51) Int. Cl. A24F 40/485; A24F 40/10; A24F 40/42; A24F 40/46

(52) U.S. Cl. CPC A24F 40/485; A24F 40/10; A24F 40/42; A24F 40/46

(57) ABSTRACT

Atomization device includes an atomization core assembly, a sealing member, and a housing. The atomization core assembly is configured to heat an atomized liquid. The housing defines a first injection hole allowing injection of the atomized liquid. The atomization core assembly and the sealing member are connected to each other and both located within the housing. The sealing member is at least partially located between the atomization core assembly and the first injection hole. The sealing member is located between the atomization core assembly and the first injection hole.

### CROSS-REFERENCE TO RELATED APPLICATIONS

This application claims priority under 35 U.S.C. § 119 (a) to and the benefit of Chinese Patent Application No. 202420250718.5, filed Jan. 31, 2024, and Chinese Patent Application No. 202420255548.X, filed Jan. 31, 2024, the entire disclosures of each of which are incorporated herein by reference.

### TECHNICAL FIELD

The disclosure relates to the field of atomization devices, and in particular, to an atomization device.

### BACKGROUND

An existing atomization device includes a main body and an atomization core assembly, where at least part of the atomization core assembly is located in an accommodating chamber defined by the main body. The atomization core assembly defines an airway therein and is configured to generate aerosol and allows the aerosol to flow through the airway to the exterior for inhalation of a user.

After prolonged use of the atomization device, the atomized liquid originally contained within the atomization core assembly will gradually be consumed. Therefore, it is usually necessary to add atomized liquid to the atomization device to prevent the atomization core assembly from dry burning. In the related art, when atomized liquid is added to the atomization device, the large flow rate of the added atomized liquid may easily cause it to impact the atomization core assembly, leading to leakage of the atomized liquid.

### SUMMARY

Embodiments of the disclosure aim to solve a technical problem that in an atomization device in the related art, atomized liquid tends to impact an atomization core assembly and cause leakage when the atomized liquid is added.

In order to solve to above technical problem, an atomization device is provided in the embodiments of the disclosure. The atomization device includes an atomization core assembly, a sealing member, and a housing. The atomization core assembly is configured to heat an atomized liquid. The housing defines a first injection hole allowing injection of the atomized liquid. The atomization core assembly and the sealing member are connected to each other and both located within the housing. The sealing member is at least partially located between the atomization core assembly and the first injection hole.

Compared with the related art, the embodiments of the disclosure mainly have following beneficial effects.

In the disclosure, the sealing member is located between the atomization core assembly and the first injection hole. Therefore, when atomized liquid is injected by a user into the atomization device through the first injection hole using an infusion member, the atomized liquid flowing out of the infusion member cannot directly contact or impact the atomization core assembly. Instead, the atomized liquid will first contact the sealing member and then gradually permeate and flow into the atomization core assembly. In summary, the atomization device in this embodiment can prevent the atomized liquid from impacting the atomization core assembly and causing leakage.

### BRIEF DESCRIPTION OF THE DRAWINGS

To describe the solutions in the disclosure or in the related art more clearly, the following briefly introduces the accompanying drawings required for describing the embodiments or the related art. Apparently, the accompanying drawings in the following illustrations show merely some embodiments of the disclosure, and those of ordinary skill in the art may still derive other accompanying drawings from these accompanying drawings without creative efforts.

FIG. 1 is a schematic structural view of an atomization device according to an embodiment of the disclosure.

FIG. 2 is a schematic structural view of an atomization device according to another embodiment of the disclosure.

FIG. 3 is an exploded schematic view of the atomization device in FIG. 1.

FIG. 4 is a top view of a support in FIG. 3.

FIG. 5 is a bottom view of a support in FIG. 3.

FIG. 6 is a schematic structural view illustrating assembly of an atomization core assembly and the support in FIG. 3.

FIG. 7 is a schematic structural view of a sealing member in FIG. 3.

FIG. 8 is a cross-sectional view of the atomization device in FIG. 1.

FIG. 9 is a schematic structural view of the atomization device in FIG. 8 when atomized liquid is added.

FIG. 10 is a schematic view of an internal structure of an atomization device according to an embodiment of the disclosure.

FIG. 11 is a schematic structural view illustrating assembly of a sealing member and an atomization core assembly according to another embodiment of the disclosure.

FIG. 12 is a schematic structural view illustrating assembly of the sealing member and the atomization core assembly in FIG. 11 and an infusion member.

FIGS. 13 to 15 are schematic views illustrating an elastic injection plug in various states according to another embodiment.

Reference signs in the accompanying drawings are described as follows:
atomization device 10, infusion member 20, housing 100, elastic injection plug 111, fixing portion 112, flipping portion 113, pressing portion 114, first injection hole 120, sealing member 200, limiting portion 210, protruding portion 220, sealing-member body 230, injection gap 240, first elastic protrusion 250, second elastic protrusion 260, atomization core assembly 300, second injection hole 311, flow channel 312, inhalation channel 313, lead 320, support 400, support body 410, first mounting hole 420, guiding portion 430, guiding surface 431, guiding channel 440, first sidewall 450, second sidewall 460, liquid absorbent fiber 500, first liquid-absorbent-fiber 510, second liquid-absorbent-fiber 520, base 600, liquid reservoir 610, electrode 700.

### DETAILED DESCRIPTION

Unless otherwise defined, all technologies and scientific terms used in the disclosure have common meanings that are able to be understood by those skilled in the art. All terms used in the disclosure are only for the purpose of illustrating specific embodiments, but are not intended to limit the disclosure. Terms “comprising”, “including”, “having” and any variants thereof are intended to cover non-exclusive inclusions. Terms “first”, “second”, etc., in the specification and claims of the disclosure and the above accompanying drawings are used to distinguish different objects, but are not used to describe a specific order.

The term “embodiment” as used herein means that specific features, structures, or characteristics described with reference to the accompanying drawings may be included in at least one embodiment of the disclosure. Phrases in the disclosure are not necessary to refer to the same embodiment and do not refer to an independent embodiment and an alternative embodiment which are exclusive to other embodiments. It may be explicitly and implicitly understood by those skilled in the art that embodiments described in the disclosure may be combined with other embodiments.

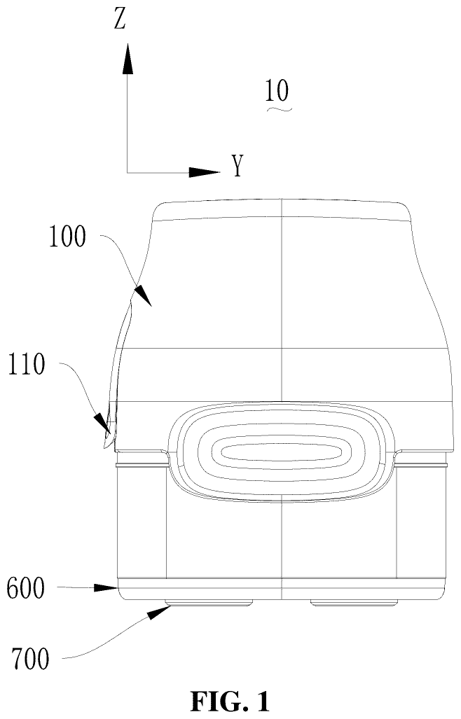

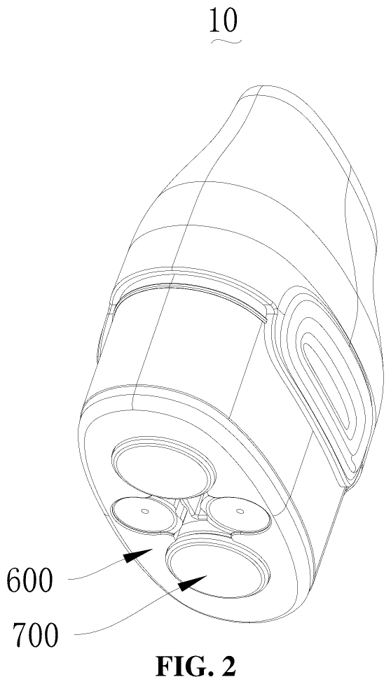

To solve the described problem, referring to FIGS. 1 to 3 and FIGS. 7 to 9, an atomization device 10 is provided in embodiments. The atomization device 10 includes an atomization core assembly 300, a sealing member 200, and a housing 100. The atomization core assembly 300 is configured to heat atomized liquid. The housing 100 defines a first injection hole 120 allowing injection of atomized liquid. The atomization core assembly 300 and the sealing member 200 are connected to each other and both located within the housing 100. The sealing member 200 is at least partially located between the atomization core assembly 300 and the first injection hole 120.

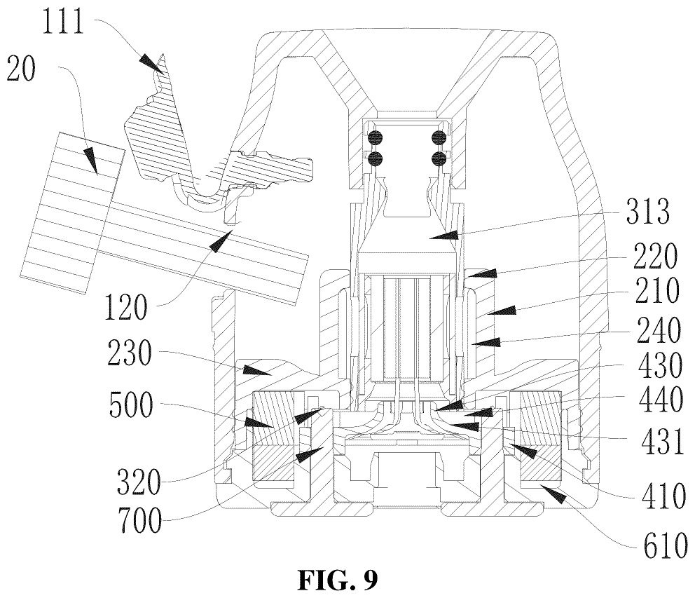

Since the sealing member 200 is located between the atomization core assembly 300 and the first injection hole 120, by the method illustrated in FIG. 9, when a user uses an infusion member 20 to inject the atomized liquid into the atomization device 10 through the first injection hole 120, the atomized liquid output from the infusion member 20 will not directly contact and impact the atomization core assembly 300, but firstly contacts the sealing member 200 and then gradually flows into the atomization core assembly 300.

Therefore, the atomization device 10 in this embodiment can avoid leakage caused by the impact of the atomized liquid to the atomization core assembly 300.

Further, if the sealing member 200 is in contact with an outer sidewall of the atomization core assembly 300, it may take a long time for the atomized liquid to contact the atomization core assembly 300, resulting in a decrease in heating efficiency. In this embodiment, the sealing member 200 is at least partially located between the atomization core assembly 300 and the first injection hole 120. An injection gap 240 is defined between at least part of the sealing member 200 and the outer sidewall of the atomization core assembly 300. The injection gap 240 allows flow of the atomized liquid. In this case, the injection gap 240 can avoid the atomized liquid from impacting the atomized core assembly 300 while improving the rate at which the atomized liquid contacts the atomization core assembly 300.

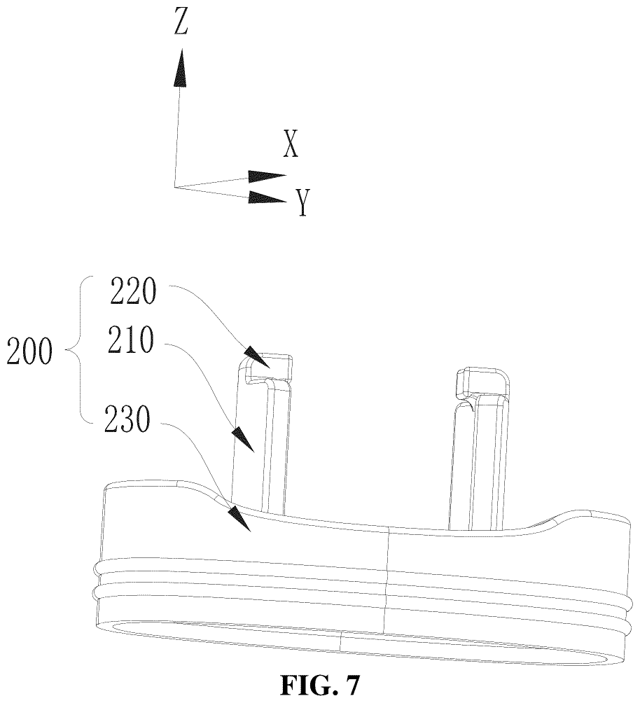

Further, referring to FIGS. 7 to 9, the sealing member 200 includes a limiting portion 210 and a protruding portion 220 connected to each other. The injection gap 240 is defined between the limiting portion 210 and the outer sidewall of the atomization core assembly 300.

The protruding portion 220 is located at one end of the limiting portion 210 close to the first injection hole 120. The protruding portion 220 protrudes towards the outer sidewall of the atomization core assembly 300 and abuts against the outer sidewall of the atomization core assembly 300.

During injection of the atomized liquid into the atomization device 10 in practical use, the atomized liquid may impact the sealing member 200 to result in deformation of the sealing member 200, and thus the effect of avoiding the atomized liquid from impacting the atomization core assembly 300 cannot be realized. In this embodiment, by disposing the protruding portion 220, the protruding portion 220 can prevent deformation of the limiting portion 210 by contacting the outer sidewall of the atomization core assembly 300 when the atomized liquid impacts the sealing member 200.

Further, a thickness of the protruding portion 220 is smaller than a thickness of the limiting portion 210 to define a communication opening (not marked in the figure) in communication with the injection gap 240. The communication opening allows the atomized liquid to flow into the injection gap 240.

It may be understood that, in some embodiments, when the sealing member 200 surrounds the sidewall of the atomization core assembly 300, or when part of the sidewall of the atomization core assembly 300 not covered by the sealing member 200 is covered by other components, the atomized liquid can only enter the injection gap 240 through the communication opening, and then enter the interior of the atomization core assembly 300. In this case, the atomized liquid can further be avoided from impacting the atomization core assembly 300. When the atomized liquid is added, the infusion member 20 can be aligned with the communication opening to enable the atomized liquid to enter the injection gap 240 through the communication opening, thereby improving the injection efficiency of the atomized liquid.

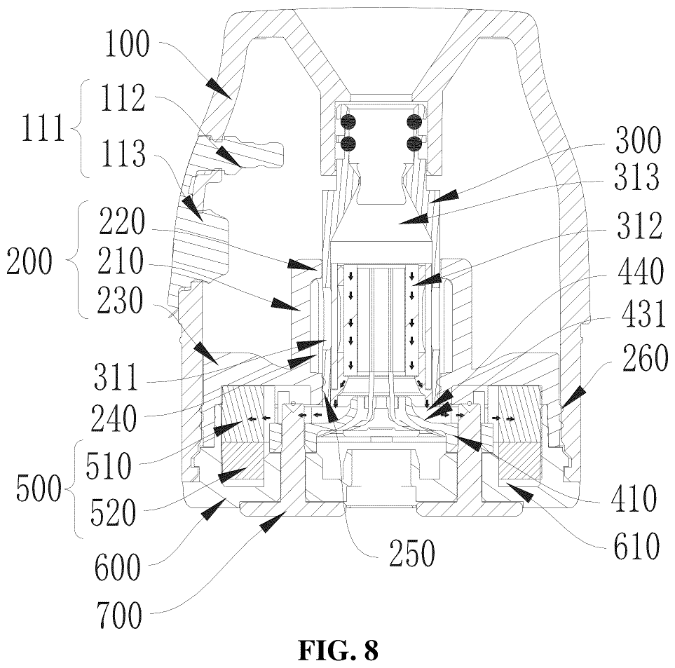

Further, referring to FIG. 7 and FIG. 8, a width of the injection gap 240 ranges from 0.5 mm to 1 mm; and/or the thickness of the protruding portion 220 ranges from 1.1 mm to 1.5 mm, and the thickness of the limiting portion 210 ranges from 0.5 mm to 1 mm; and/or the injection gap 240 gradually increases in width in a direction away from the protruding portion 220; and/or a height of the first injection hole 120 is equal to a height of the injection gap 240.

In FIG. 7, a height direction is Z direction, and a width direction is Y direction.

When the width of the injection gap 240 is 0.5 mm-1 mm, the atomized liquid can form a gap liquid-flow phenomenon in the injection gap 240. The liquid flow is caused by a pure pressure difference, that is, the pressure-difference flow. This can avoid ineffective obstruction of the atomized liquid impact on the atomization core assembly 300 due to an overly wide injection gap 240 and also allows the atomized liquid to ascend along the injection gap 240, facilitating the atomized liquid to enter the interior of the atomization core assembly 300, along the injection gap 240, through the second injection hole 311 on the outer sidewall of the atomization core assembly 300.

The thickness of the protruding portion 220 needs to be larger than the thickness of the limiting portion 210, so that when the protruding portion 220 abuts against the outer sidewall of the atomization core assembly 300, the injection gap 240 can be defined between the limiting portion 210 and the outer sidewall of the atomization core assembly 300. The difference between the thickness of the protruding portion 220 and the thickness of the limiting portion 210 determines the size of the communication opening, that is, determines the rate at which the atomized liquid enters the injection gap 240. Therefore, in the range of the thickness of the protruding portion 220 and the thickness of the limiting portion 210 in the disclosure, the atomized liquid can easily enter the injection gap 240, and liquid leakage can be avoided, where the liquid leakage is caused by excessive impact applied to the atomization core assembly 300 due to injection of too much atomized liquid at one time.

It can be understood that, the width of the injection gap 240 may be any one of 0.5 mm, 0.6 mm, 0.7 mm, 0.8 mm, 0.9 mm, and 1 mm, or in a range formed by any two of 0.5 mm, 0.6 mm, 0.7 mm, 0.8 mm, 0.9 mm, and 1 mm. The thickness of the protruding portion may be any one of 1 mm, 1.1 mm, 1.2 mm, 1.3 mm, 1.4 mm, and 1.5 mm, or in a range formed by any two of 1 mm, 1.1 mm, 1.2 mm, 1.3 mm, 1.4 mm, and 1.5 mm. The thickness of the limiting portion may be any one of 0.5 mm, 0.6 mm, 0.7 mm, 0.8 mm, 0.9 mm, and 1 mm, or in a range formed by any two of 0.5 mm, 0.6 mm, 0.7 mm, 0.8 mm, 0.9 mm, and 1 mm. Regarding the injection gap 240, the width and volume of the injection gap can be increased by defining a groove on a surface of the limiting portion 210 facing the atomization core body 300. Preferably, when the thickness of the protruding portion 220 is 1.3 mm, the thickness of the limiting portion 210 is 0.8 mm, and the width of the injection gap 240 is 0.85 mm, the atomization device 10 has the desired performance, where the atomized liquid can easily enter the injection gap 240, and the liquid leakage, caused by excessive impact applied to the atomization core assembly 300 due to injection of too much atomized liquid at one time, can be avoided.

Further, in the case where the injection gap 240 gradually increases in width in the direction away from the protruding portion 220, when the user inhales the atomization device 10, the atomized liquid may be moved from bottom to top into the atomization core assembly 300. In this case, the shape of the injection gap 240 can ensure that the space for the atomized liquid to move gradually decreases, thereby improving the tension of the atomized liquid and the adhesion of the atomized liquid to the limiting portion 210 and the atomization core assembly 300 are improved, thereby avoiding the atomized liquid from not being unable drawn and transported.

Further, if the height of the first injection hole 120 is equal to the height of the injection gap 240, after the user inserts the infusion member 20 into the first injection hole 120, a liquid outlet (not marked in the figure) of the infusion member 20 will be located just above the injection gap 240, thereby facilitating the atomized liquid to enter the injection gap 240 through the communication opening.

Further, referring to FIGS. 7 to 9, the sealing member 200 further includes a sealing-member body 230. The sealing-member body 230 defines a fixing hole (not illustrated in the figure). The atomization core assembly 300 is located in the fixing hole, and is fixedly connected to the sealing-member body 230. The limiting portion 210 is disposed at an outer periphery of the fixing hole.

In this case, the sealing member 200 can be tightly connected to the atomization core assembly 300 to prevent the atomization core assembly 300 and the sealing member 200 from slipping relative to each other, thereby preventing the sealing member 200 from being unable to continuously alleviate the impact of the atomized liquid.

Further, referring to FIG. 8 and FIG. 9, the sealing-member body 230 includes a first sealing-member body (not marked in the figure) and a second sealing-member body (not marked in the figure) that are connected to each other. The first sealing-member body is close to the inner sidewall of the housing 100, and the second sealing-member body is close to the atomization core assembly 300 and is connected to the protruding portion 220. The height of the first sealing-member body is larger than the height of the second sealing-member body.

In this case, the height direction is a direction of a connection line from the center of the base 600 to the center of the atomization core assembly 300. Since the height of the first sealing-member body is larger than the height of the second sealing-member body, the atomized liquid can flow from the inner sidewall of the housing 100 towards the atomization core assembly 300, so that when the atomization device 10 has less atomized liquid, the atomized liquid can still naturally flow towards the atomization core assembly 300.

It can be understood that, as illustrated in FIG. 8 and FIG. 9, the second sealing-member body may have an inclined surface, and in this case, the atomized liquid can smoothly flow along the inclined surface. Alternatively, there may be a height difference between the first sealing-member body and the second sealing-member body, and in this case, the first sealing-member body and the second sealing-member body form a step-like shape, and the atomized liquid can still have a tendency to flow towards the atomization core assembly 300 due to the viscosity and fluidity of the atomized liquid.

Further, referring to FIGS. 7 to 9, there are multiple limiting portions 210 and multiple protruding portions 220, and the multiple limiting portions 210 is in a one-to-one correspondence with the multiple protruding portions 220. The multiple limiting portions 210 are symmetrically and circumferentially distributed along the outer periphery of the fixing hole.

In this case, the limiting portion 210 and the protruding portion 220 can clamp the atomization core assembly 300. Due to the symmetrical distribution of the multiple limiting portions 210 and the multiple protruding portions 220, the atomization core assembly 300 is evenly stressed, so that the stability of the atomization core assembly 300 can be improved.

Further, referring to FIGS. 7 to 9, a first elastic protrusion 250 is disposed on an inner sidewall of the fixing hole. The first elastic protrusion 250 abuts against the outer sidewall of the atomization core assembly 300 to enable the atomization core assembly 300 to be in an interference fit with the fixing hole.

In this case, the interference fit between the atomization core assembly 300 and the fixing hole can further prevent the relative slipping between the atomization core assembly 300 and the sealing member 200.

Further, the first elastic protrusion 250 is an annular protrusion; and/or there are multiple first elastic protrusions 250, and the multiple first elastic protrusions 250 are distributed in an axial direction of the fixing hole; and/or a second elastic protrusion 260 is further disposed on an outer sidewall of the sealing-member body 230, and the second elastic protrusion 260 abuts against the inner sidewall of the housing 100 to enable the sealing-member body 230 to be in interference fit with the housing 100.

When the first elastic protrusion 250 is an annular protrusion and/or there are multiple first electric protrusions, the outer sidewall of the atomization core assembly 300 is subjected to uniform forces at various positions, and the stability of the atomization core assembly 300 is stronger. Similarly, the second elastic protrusion 260 can strengthen structural strength between the sealing member 200 and the housing 100, and further prevent the sealing member 200 from slipping.

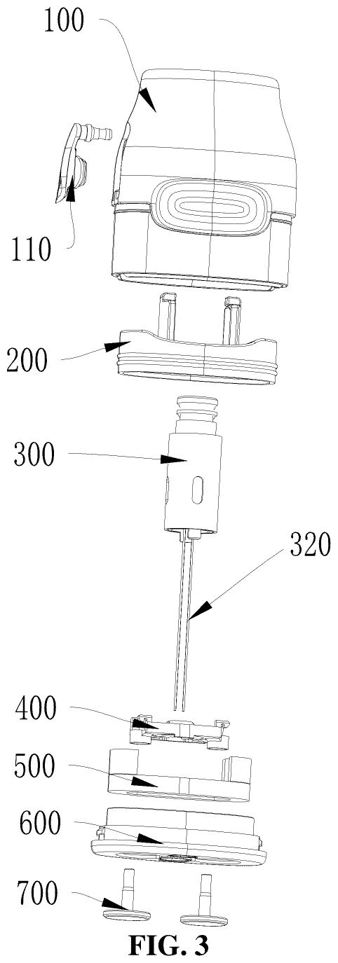

Further, referring to FIG. 3 and FIGS. 7 to 9, the outer sidewall of the atomization core assembly 300 defines a second injection hole 311. The second injection hole 311 allows the atomized liquid to flow into the interior of the atomization core assembly 300 from the injection gap 240. An orthographic projection of the limiting portion 210 on the outer sidewall of the atomization core assembly 300 covers at least part of the second injection hole 311.

Generally, the second injection hole 311 allows the atomized liquid to enter the interior of the atomization core assembly 300 from the injection gap 240, so that in order to avoid the atomized liquid from entering the interior of the atomization core assembly 300 directly from the injection gap 240, in the embodiments, the limiting portion 210 covers the second injection hole 311. In this case, if the atomized liquid intends to impact the atomization core assembly 300 and enter the interior of the atomization core assembly 300 directly, the atomized liquid is bound to be buffered by the limiting portion 210. In addition, a material of the limiting portion 210 corresponding to the part of the outer sidewall of the atomization core assembly 300 that does not define the second injection hole 311 can be saved, thereby reducing the production cost of the atomization device 10.

Further, referring to FIGS. 1 to 3, FIG. 8, and FIG. 9, a width direction of the housing 100 is Y direction in FIG. 1. The width of the housing 100 is larger than the thickness of the housing 100. Two symmetrically distributed first accommodating cavities (not marked in the figure) and two symmetrically distributed second accommodating cavities (not marked in the figure) are defined between the atomization core and the inner sidewall of the housing 100. The first accommodating cavity is larger than the second accommodating cavity. The first injection hole 120 and the limiting portion 210 are both located in the first accommodating cavity.

In practical production of the atomization device 10, the housing 100 of the atomization device 10 may be set in an elliptic shape. In this case, the first injection hole 120 may be defined at one side of the housing 100 away from a central point of the housing 100, that is, one side where the first accommodating cavity is located.

In this case, the atomization core assembly 300 is covered by the housing 100 at both sides of the second accommodation cavity, and the gap between the atomization core assembly 300 and the housing 100 is relatively small. Even if the atomization core assembly 300 defines the second injection hole 311 on a sidewall of the second accommodation cavity, it is difficult for the atomized liquid to impact part of the atomization core assembly 300 in the second accommodation cavity after the atomized liquid is injected by the infusion member 20. Therefore, it is unnecessary to dispose the protruding portion 220 in the second accommodating cavity, thereby reducing the production cost of the atomization device 10.

Further, referring to FIG. 1, FIG. 8, and FIG. 9, the housing 100 is further provided with an elastic injection plug 111 corresponding to the first injection hole 120. The elastic injection plug 111 includes a fixing portion 112 and a flipping portion 113. The fixing portion 112 is fixedly connected to the housing 100. The flipping portion 113 is connected to the fixing portion 112 and is detachably connected to the first injection hole 120 and is flippable to block the first injection hole 120 or communicate the first injection hole with the infusion member 20.

In this case, in the atomization device 10 in this embodiment, the elastic injection plug 111 can be flippable, so that the elastic injection plug 111 is deformed to block the first injection hole 120 or communicate the first injection hole 120 with the infusion member 20. Thus, the injection efficiency of the atomized liquid in the atomization device 10 is improved.

When the atomization device 10 is used, the atomized liquid is heated to form aerosol, and the condensate formed by condensation of the atomized liquid and the aerosol in the atomization device 10 will generally accumulate at the bottom of the atomization device 10. In the related art, the random accumulation and flow of the atomized liquid and the condensate often results in liquid leakage of the atomization device 10, which affects the experience of the user and also reduces the service life of the atomization device 10.

A liquid reservoir 610 may be a cavity or a groove separately defined by the base 600, or a cavity defined by the liquid reservoir 610 and other components in the atomization device 10 together.

To solve the described technical problem, reference can be made to FIGS. 1 to 5 and FIGS. 8 to 10. An atomization device 10 is provided in an embodiment of the disclosure. The atomization device 10 includes an atomization core assembly 300, a support 400, and a base 600. A flow channel 312 is defined in the atomization core assembly 300. The flow channel 312 allows flow of liquid. The support 400 includes a support body 410 and a guiding portion 430. The guiding portion 430 protrudes from the support body 410 and is connected to the bottom of the atomization core assembly 300. An outer sidewall of the guiding portion 430 has a guiding surface 431. The guiding surface 431 is an inclined surface or an arc surface. A perimeter of the guiding portion 430 gradually decreases in a direction in which the guiding portion 430 protrudes. The base 600 defines a liquid reservoir 610. Part of the support body 410 is located in the liquid reservoir 610. The guiding surface 431 and a surface of the support body 410 facing the atomization core assembly 300 defines a guiding channel 440. Two ends of the guiding channel 440 are respectively in communication with the flow channel 312 and the liquid reservoir 610.

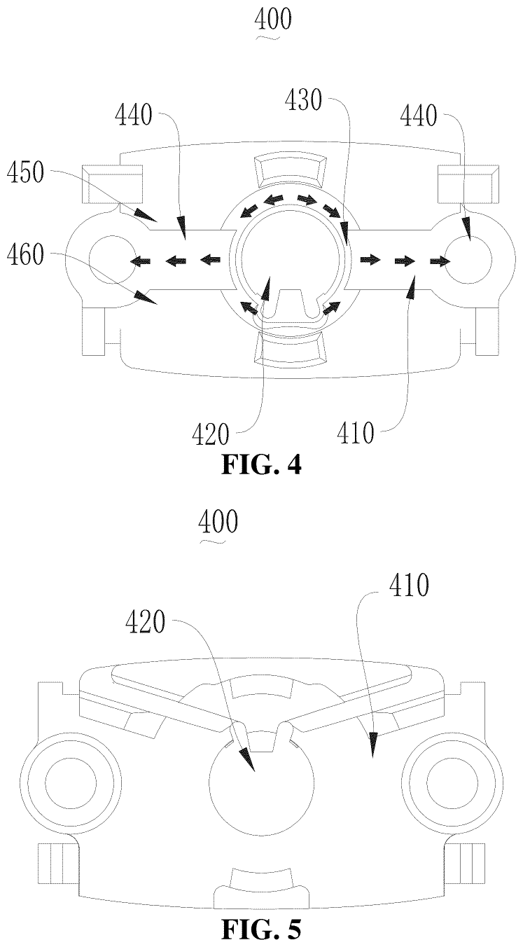

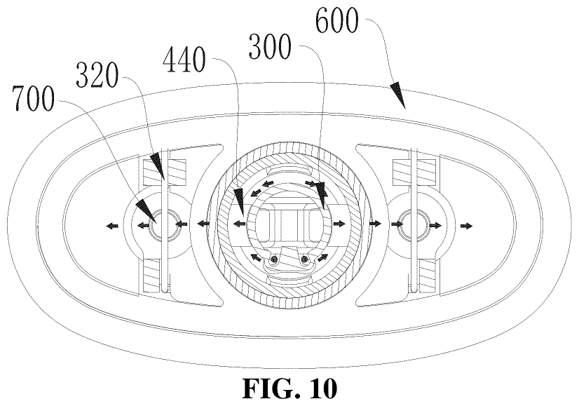

The directions indicated by arrows in FIG. 4, FIG. 8, and FIG. 10 are flow directions of the atomized liquid and the condensate. It can be seen from the figures that the atomized liquid and the condensate flow from top to bottom inside the atomization core assembly 300 onto the guiding surface 431, and then since the guiding surface 431 is the inclined surface or the arc surface, the perimeter of the guiding portion 430 gradually decreases in the direction in which the guiding portion 430 protrudes, thus the guiding surface 431 can guide the atomized liquid and the condensate to smoothly flow from the guiding portion 430 to a side edge of the support body 410. Since part of the support body 410 is located in the liquid reservoir 610, the thermal material to-be-heated and the condensate can smoothly flow to the side edge of the support body 410 and then flow from the side edge of the support body 410 into the liquid reservoir 610. Therefore, in this embodiment, the purpose of gathering the atomized liquid and the condensate in the liquid reservoir 610 is realized, thereby successfully avoiding the atomized liquid and the condensate from randomly accumulating and flowing in the atomization device 10.

Further, referring to FIG. 3, FIG. 8, and FIG. 9, the liquid absorbent fiber 500 is disposed in the liquid reservoir 610. The guiding channel 440 is in communication with the liquid absorbent fiber 500.

The liquid absorbent fiber 500 includes components such as liquid absorbent cotton, and can absorb the atomized liquid and the condensate. In this case, the liquid absorbent fiber 500 can prevent the atomized liquid and the condensate from overflowing when the atomization device 10 shakes, thereby further preventing leakage of the atomized liquid and the condensate.

Further, referring to FIG. 3, FIG. 8, and FIG. 9, a fixing groove is defined at the bottom of the liquid reservoir 610. The liquid absorbent fiber 500 includes first liquid-absorbent-fiber 510 and second liquid-absorbent-fiber 520 stacked with the first liquid-absorbent-fiber 510. The second liquid-absorbent-fiber 520 is snapped into the fixing groove. The first liquid-absorbent-fiber 510 is snapped between the second liquid-absorbent-fiber 520 and a top wall of the liquid reservoir 610.

In this case, the absorption amount of the atomized liquid and the condensate can be improved by means of the double-layer liquid absorbent fiber 500. In addition, the first liquid-absorbent-fiber 510 is snapped between the second liquid-absorbent-fiber 520 and the top wall of the liquid reservoir 610, so that there is no need to additionally dispose a structure to fix the first liquid-absorbent-fiber 510. Therefore, the inner space of the atomization device 10 is fully used, the space utilization rate of the atomization device 10 is further improved, and the production cost of the atomization device 10 is reduced.

Further, referring to FIG. 8 and FIG. 9, the guiding channel 440 is in communication with the first liquid-absorbent-fiber 510; and/or an outer sidewall of the support body 410 and sidewalls of both the first liquid-absorbent-fiber 510 and the second liquid-absorbent-fiber 520 defines a liquid absorbent gap.

The atomized liquid and the condensate stored in the liquid reservoir 610 can climb up to the interior of the atomization core assembly 300 again under the inhalation of the user, and are heated for the second time to form aerosol. If the liquid to-be-heated is stored in the second liquid-absorbent-fiber 520 but not in the first liquid-absorbent-fiber 510, it is difficult for the atomized liquid and the condensate to continuously climb into the atomization core assembly 300.

In the embodiments, since the guiding channel 440 is in communication with the first liquid-absorbent-fiber 510, after the atomized liquid and the condensate flow out through the side edge of the support body 410, the atomized liquid and the condensate flow to the first liquid-absorbent-fiber 510 first. After the first liquid-absorbent-fiber 510 is fully stored, the atomized liquid and the condensate gradually flow to the second liquid-absorbent-fiber 520. Therefore, a situation where the first liquid-absorbent-fiber 510 does not store liquid while the second liquid-absorbent-fiber 520 stores liquid can be avoided.

Further, referring to FIG. 8 and FIG. 9, the second liquid-absorbent-fiber 520 is located between the support body 410 and the base 600. The support body 410 defines a guiding hole. The guiding hole extends through the support body 410, and has one end in communication with an upper surface of the second liquid-absorbent-fiber 520.

In some embodiments, the support body 410 may define a guiding hole (not marked in the figure). The storage of the atomized liquid and the condensate can be speeded up through the guiding hole.

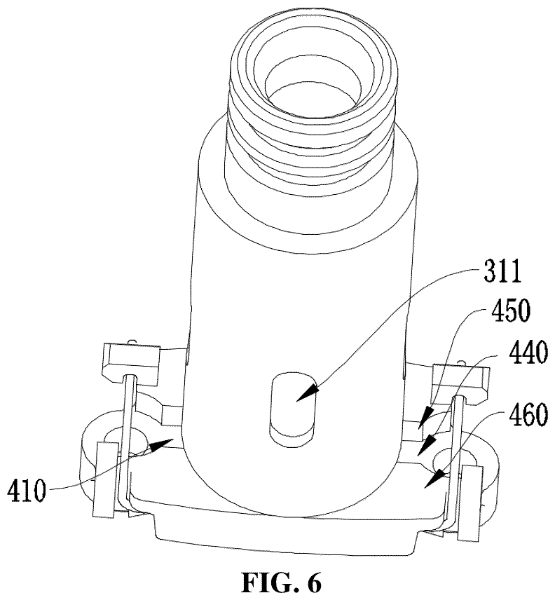

Further, referring to FIGS. 4 to 10, the support 400 further has a first sidewall 450 and a second sidewall 460. The guiding portion 430, the first sidewall 450, the second sidewall 460, and an upper surface of the support body 410 cooperatively define the guiding channel 440.

In this case, the guiding channel 440 is in the shape of a sliding groove, and can avoid the atomized liquid and the condensate from flowing from the atomization core assembly 300 to a portion where the guiding channel 440 is not defined. In other words, the atomized liquid and the condensate are limited to flow in a certain shape and path, thereby further preventing leakage of the atomized liquid and the condensate.

Further, referring to FIGS. 8 to 12, the atomization device 10 further includes a liquid absorbent member. The liquid absorbent member has one end located in the guiding channel 440, and the other end located in the flow channel 312.

In some cases, the user needs to inhale the atomized liquid and the condensate in the liquid reservoir 610 or the guiding channel 440 into the interior of the atomization core assembly 300, and flowing of liquid needs to depend on solids. Therefore, in this embodiment, the liquid absorbent member is additionally disposed, one end of the liquid absorbent member is located in the guiding channel 440, and the other end of the liquid absorbent member is located in the flow channel 312. In this case, the liquid can climb along the liquid absorbent member, thereby entering the flow channel 312 from the guiding channel 440.

Further, referring to FIG. 8 and FIG. 9, the atomization device 10 further includes a housing 100 and a sealing member 200. The base 600 is connected to the bottom of the housing 100. The sealing member 200 is located in the housing 100 and is connected to an upper surface of the base 600. The sealing member 200 and the upper surface of the base 600 define a liquid reservoir 610.

In this case, the liquid reservoir 610 is cooperatively defined by the sealing member 200 and the base 600. The size of the liquid reservoir can vary with a distance between the sealing member 200 and the base 600.

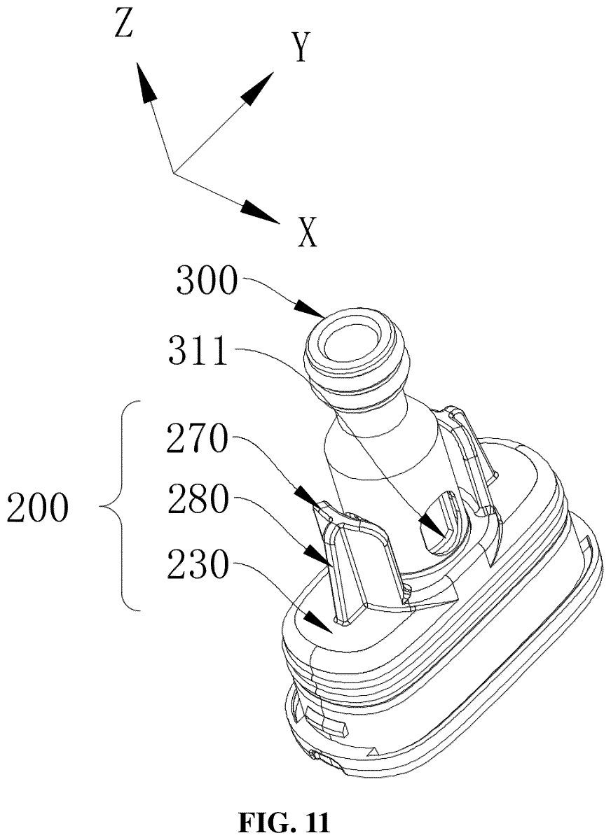

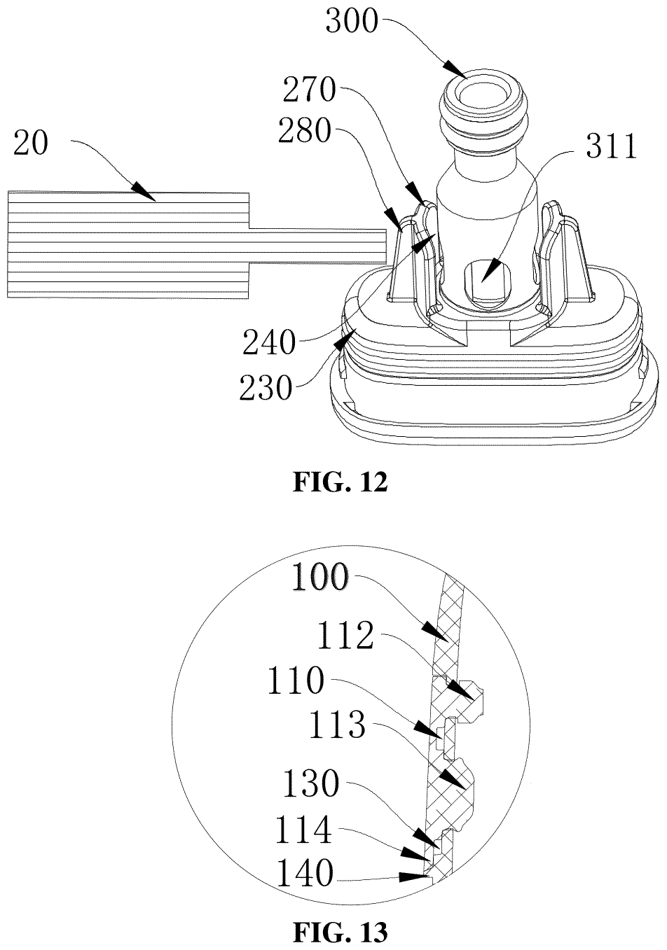

Referring to FIGS. 11 and 12, FIGS. 11 and 12 illustrate the sealing member 200 provided in another embodiment of the disclosure. The sealing member 200 is located between the first injection hole and the atomization core assembly 300, and the sealing member 200 includes a blocking portion 270 and a shunting portion 280.

The blocking portion 270 is closer to the atomization core assembly 300 than the shunting portion 280 and surrounds at least part of the sidewall of the atomization core assembly 300. The shunting portion 280 is located on one side of the blocking portion 270 close to the first injection hole and protrudes toward the first injection hole.

Specifically, since the sealing member 200 is located between the atomization core assembly 300 and the first injection hole, the atomized liquid flows through the sealing member 200 and then gradually flows to the atomization core assembly 300. Thus, the atomization core assembly 300 can avoid being impacted by the atomized liquid, thereby preventing the atomization device 10 from leaking oil.

When the atomized liquid is injected into the atomization device 10 through the first injection hole by using the infusion member 20, the atomized liquid output from the infusion member 20 will not directly impact the atomization core assembly 300 but first contacts the shunting portion 280, which divides it into at least two streams of atomized liquid. This reduces the impact force of the atomized liquid output from the infusion member 20, makes it evenly distributed, and then the two streams of atomized liquid contact the blocking portion 270, flow through the blocking portion 270, and finally contact the atomization core assembly 300. Additionally, since the blocking portion 270 and the shunting portion 280 block the flow of the atomized liquid in the Y direction and the X direction illustrated in FIG. 11, the atomization device 10 in this embodiment can also prevent the atomized liquid from shaking inside it, thereby improving the structural stability of the atomization device 10. In summary, the atomization device 10 in this embodiment can prevent leakage and improve structural stability.

It should be understood that the shunting portion 280 and the blocking portion 270 can be integrally formed, detachably connected, or exist independently. When the shunting portion 280 is connected to the blocking portion 270 or integrally formed with the blocking portion 270, the shunting portion 280 can enhance the strength of the blocking portion 270.

Further, the shunting portion 280 can be located at a middle of the blocking portion 270 to divide the blocking portion 270 into two symmetrical blocking sub-portions (not marked in the figure). In this case, since the two blocking sub-portions are symmetrical, the atomized liquid is divided into two streams flowing along the blocking portion towards the atomization core assembly 300, with similar flow rates of the two streams of atomized liquid. This ensures equal contact areas between the atomization core assembly 300 and the atomized liquid at various points, improving the heating efficiency and the uniformity of the aerosol generated by heating, thereby enhancing the user's inhalation experience.

Further, referring to FIGS. 11 and 12, a thickness of the shunting portion 280 is smaller than a diameter of the first injection hole. An orthographic projection of at least part of the shunting portion 280 on the blocking portion 270 is located within an orthographic projection of the first injection hole on the blocking portion 270.

Specifically, a thickness direction of the shunting portion 280 is the X direction in FIG. 11. The orthographic projection should be understood as a projection range of the first injection hole along its central axis on the blocking portion 270. Since the thickness of the shunting portion 280 is smaller than the diameter of the first injection hole, and the orthographic projection of the at least part of the shunting portion 280 on the blocking portion 270 is located within the orthographic projection of the first injection hole on the blocking portion 270, a diameter of the liquid outlet of the infusion member 20 (not marked in the figure) is also larger than the shunting portion 280. When the infusion member 20 is inserted into the first injection hole and the liquid outlet abuts against the shunting portion 280, the atomized liquid can still flow out from the liquid outlet and will not be blocked by the shunting portion 280.

In summary, the shunting portion 280 in this embodiment can prevent the infusion member 20 from directly abutting against the blocking portion 270, which would otherwise completely seal the liquid outlet by the blocking portion 270. Compared with the related art without the sealing member 200, this embodiment can also prevent the liquid outlet from being sealed by the sidewall of the atomization core assembly 300.

Further, referring to FIGS. 11 and 12, at least part of the sealing member 200 and the outer sidewall of the atomization core assembly 300 cooperatively define the injection gap 240. The injection gap 240 allows the flow of the atomized liquid.

Specifically, if the sealing member 200 is in contact with the outer sidewall of the atomization core assembly 300, it may take a long time for the atomized liquid to contact the atomization core assembly 300, reducing the atomized liquid generation efficiency of the atomization device 10. In this embodiment, the sealing member 200 is at least partially located between the atomization core assembly 300 and the first injection hole. The injection gap 240 is defined between at least part of the sealing member 200 and the outer sidewall of the atomization core assembly 300. The injection gap 240 allows the flow of the atomized liquid. In this case, the injection gap 240 can avoid the atomized liquid from impacting the atomization core assembly 300 while improving the rate at which the atomized liquid contacts the atomization core assembly 300.

Further, referring to FIGS. 11 and 12, a width direction of the injection gap 240 is the Y direction in FIG. 11. The width of the injection gap 240 ranges from 0.5 mm to 1.2 mm.

Specifically, the sidewall of the atomization core assembly 300 defines the second injection hole 311. The atomized liquid needs to flow through the second injection hole 311 into the interior of the atomization core assembly 300. That is, when the user inhales the atomization device 10, the atomized liquid moves from bottom to top to the second injection hole 311 and then enters the atomization core assembly 300 for heating to generate aerosol. When the width of the injection gap 240 ranges from 0.5 mm to 1.2 mm, the atomized liquid can form the gap liquid-flow phenomenon in the injection gap 240. The liquid flow is caused by the pure pressure difference, that is, the pressure-difference flow. This can avoid ineffective obstruction of the atomized liquid impact on the atomization core assembly 300 due to an overly wide injection gap 240 and also allows the atomized liquid to ascend along the injection gap 240, facilitating the atomized liquid to enter the interior of the atomization core assembly 300, along the injection gap 240, through the second injection hole 311 on the outer sidewall of the atomization core assembly 300.

It can be understood that the width of the injection gap 240 may be any one of 0.5 mm, 0.6 mm, 0.7 mm, 0.8 mm, 0.9 mm, 1.0 mm, 1.1 mm, and 1.2 mm, or in a range formed by any two of 0.5 mm, 0.6 mm, 0.7 mm, 0.8 mm, 0.9 mm, and 1 mm.

Further, referring to FIGS. 11 and 12, the injection gap 240 gradually increases in width in a direction from the bottom of the atomization core assembly 300 to the top of the atomization core assembly 300.

Specifically, in the case where the injection gap 240 gradually increases in width in the direction from the bottom to the top of the atomization core assembly 300, when the user inhales the atomization device 10, the shape of the injection gap 240 can ensure that the space for the atomized liquid to move gradually decreases, thereby improving the tension of the atomized liquid and the adhesion of the atomized liquid to the sealing member 200 and the atomization core assembly 300, preventing the atomized liquid from not being drawn and transported.

Further, referring to FIGS. 11 and 12, the sealing member 200 is implemented as multiple sealing members that are symmetrically and circumferentially distributed along an outer periphery of the atomization core assembly 300.

Specifically, in this case, the multiple sealing members 200 can clamp the atomization core assembly 300. The symmetrical distribution of the multiple sealing members 200 can ensure that the atomization core assembly 300 is evenly stressed, so that the stability of the atomization core assembly 300 can be improved.

Further, referring to FIGS. 11 and 12, the sealing member 200 is elastic.

Specifically, in this case, when the infusion member 20 is pressed against the sealing member 200 with a certain force, the sealing member 200 can deform and fit the sidewall of the atomization core assembly 300, thus protecting the sidewall of the atomization core assembly 300 from deformation and further preventing the atomized liquid from impacting the atomization core assembly 300.

Further, referring to FIGS. 11 and 12, the sealing member 200 further includes the sealing-member body 230. The sealing-member body 230 defines a fixing hole (not marked in the figure), the atomization core assembly 300 is located in the fixing hole and fixedly connected to the sealing-member body 230. The blocking portion 270 is disposed at the outer periphery of the fixing hole.

In this case, the sealing member 200 can be tightly connected to the atomization core assembly 300 to prevent the atomization core assembly 300 and the sealing member 200 from slipping relative to each other, thereby preventing the sealing member 200 from being unable to continuously alleviate the impact of the atomized liquid.

Further, the sealing-member body 230 includes the first sealing-member body 230 and the second sealing-member body 230 connected to each other. The first sealing-member body 230 is close to the inner sidewall of the housing 100, the second sealing-member body 230 is close to the atomization core assembly 300 and is connected to the blocking portion 270. The height of the first sealing-member body 230 is greater than the height of the second sealing-member body 230.

Further, referring to FIG. 11 and FIG. 12, the sealing-member body 230 includes a first sealing-member body (not marked in the figure) and a second sealing-member body (not marked in the figure) that are connected to each other. The first sealing-member body is close to the inner sidewall of the housing 100, and the second sealing-member body is close to the atomization core assembly 300 and is connected to the blocking portion 270. The height of the first sealing-member body is larger than the height of the second sealing-member body.

In this case, the height direction is Z direction in FIG. 11. Since the height of the first sealing-member body is larger than the height of the second sealing-member body, the atomized liquid can flow from the inner sidewall of the housing 100 towards the atomization core assembly 300, so that when the atomization device 10 has less atomized liquid, the atomized liquid can still naturally flow towards the atomization core assembly 300 under the action of gravity.

It can be understood that, as illustrated in FIG. 11 and FIG. 12, the second sealing-member body may have an inclined surface, and in this case, the atomized liquid can smoothly flow along the inclined surface. Alternatively, there may be a height difference between the first sealing-member body and the second sealing-member body, and in this case, the first sealing-member body and the second sealing-member body form a step-like shape, and the atomized liquid can still have a tendency to flow towards the atomization core assembly 300 due to the viscosity and fluidity of the atomized liquid.

Further, referring to FIGS. 4 to 6 and FIGS. 8 to 10, a first mounting hole 420 is disposed at the middle of the guiding portion 430. The bottom of the atomization core assembly 300 covers the first mounting hole 420. The atomization core assembly 300 further includes a lead 320. The lead 320 passes through the first mounting hole 420.

In this case, the atomization core assembly 300 covers the first mounting hole 420, so that the lead 320 can be avoided from being bumped, and the atomized liquid and the condensate can be avoided from flowing into the first mounting hole 420.

Further, an inhalation channel 313 is defined at the top of the atomization core assembly 300, and allows the aerosol to pass through. The inhalation channel 313 is in communication with the flow channel 312 and the external environment. A flow area of the inhalation channel 313 gradually decreases in a direction from the flow channel 312 to the external environment.

When the atomization core assembly 300 in the atomization device 10 heats the atomized liquid to produce aerosol, the aerosol will flow out to the external environment through the inhalation channel 313. Part of the aerosol will condense to form condensate due to the contact with the sidewall of the inhalation channel 313 or the temperature decrease. In this embodiment of the disclosure, the flow area of the inhalation channel 313 gradually decreases, so that the volume of the aerosol is reduced and compressed in the outward flow process, and the flow rate of aerosol in contact with the sidewall increases, so that the condensate is more easily formed, thereby realizing the repeated use of the atomization device 10.

In some embodiments, referring to FIGS. 1 to 3, the assembly of the atomization device 10 involves first installing the atomization core assembly 300 to the support 400. Then, place an oil-absorbing fiber 500 on the base 600, mount the support 400 onto the base 600, and pass the electrode 700 through the base 600, the oil-absorbing fiber 500, and the support 400 to connect with the atomization core assembly 300. Next, insert the elastic injection plug 111 into the housing 100 (which may serve as a mouthpiece) and sleeve the sealing member 200 on the atomization core assembly 300. Finally, connect the housing 100 and the base 600, encapsulating the remaining components and completing the assembly of the atomization device 10.

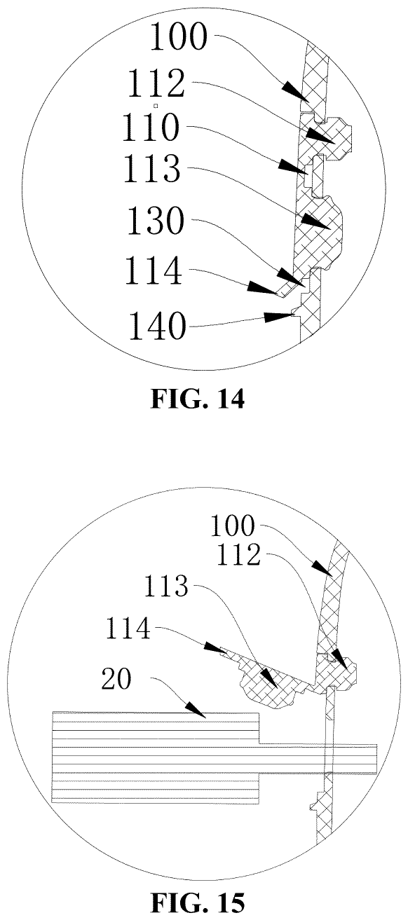

Further, in another embodiment of the disclosure, the elastic injection plug 111 corresponding to the first injection hole on the housing 100 is illustrated in FIGS. 13 to 15. The elastic injection plug 111 includes the fixing portion 112 and the flipping portion 113. The fixing portion 112 is connected to the housing 100. The flipping portion 113 is connected to the fixing portion 112. The flipping portion 113 is detachably connected to the first injection hole and is flippable to block the first injection hole or communicate the first injection hole with the infusion member 20.

Specifically, in the atomization device 10 in this embodiment, the elastic injection plug 111 can be flippable, so that the elastic injection plug 111 is deformed to block the first injection hole 120 or communicate the first injection hole 120 with the infusion member 20. Thus, the injection efficiency of the atomized liquid in the atomization device 10 is improved.

Further, referring to FIGS. 13 to 15, the housing 100 defines a mounting groove 110 and a limiting portion 140. The fixing portion 112 and the flipping portion 113 are located in the mounting groove 110. The flipping portion 113 has a pressing portion 114 at one end of the flipping portion 113 away from the fixing portion 112. The limiting portion 140 is located at one end of an outer periphery of the mounting groove 110 close to the pressing portion 114. When the pressing portion 114 abuts against the sidewall of the limiting portion 140, the pressing portion 114 and a bottom wall of the mounting groove 110 cooperatively define a deformation chamber 130. A sidewall of the limiting portion 140 and the sidewall of the mounting groove 110 cooperatively define a limiting chamber (not marked in the figure), and a sidewall of the elastic injection plug 111 is located within the limiting chamber.

Specifically, to prevent accidental triggering of the elastic injection plug 111 leading to the opening of the first injection hole, the atomization device 10 needs further improvement. In this embodiment, when the limiting portion 140 abuts against the pressing portion 114, the sidewall of the elastic injection plug 111 is located within the limiting chamber defined by the sidewall of the limiting portion 140 and the sidewall of the mounting groove 110. This means the elastic injection plug 111 is positioned inside the housing 100, making it impossible for the user to grip or pry out the elastic injection plug 111, thereby preventing accidental triggering and opening of the elastic injection plug 111.

The specific steps to open the elastic injection plug 111 in this embodiment are as follows.

First, press the pressing portion 114 towards the deformation chamber 130. At this time, the pressing portion 114 bends within the deformation chamber 130 and tilts in a direction away from the housing 100, allowing the user to grip the pressing portion 114. Then, by gripping the pressing portion 114, the flipping portion 113 can leave the first injection hole, enabling the first injection hole to be opened. Subsequently, insert the infusion member 20 into the first injection hole and infuse the atomized liquid.

Further, referring to FIG. 11 and FIG. 12, the width direction of the housing 100 is the Y direction in FIG. 11, and the thickness direction of the housing 100 is the X direction in FIG. 11. The width of the housing 100 is greater than the thickness of housing 100, the atomization core assembly 300 and the inner sidewall of the housing 100 cooperatively define the two symmetrically distributed first accommodating chambers (not marked in the figure) and the two symmetrically distributed second accommodating chambers (not marked in the figure).

The first accommodating chamber is larger than the second accommodating chamber, and both the first injection hole and the sealing member are located in the first accommodating chamber.

In the actual production of the atomization device 10, the housing 100 of the atomization device 10 may be set in an elliptical shape. In this case, the first injection hole may be defined at one side of the housing 100 away from the center point of the housing 100, that is, one side where the first accommodating chamber is located.

In this case, the atomization core assembly 300 is covered by the housing 100 at both sides of the second accommodating chamber, and the gap between the atomization core assembly 300 and the housing 100 is relatively small. Even if the atomization core assembly 300 defines the second injection hole 310 on the sidewall of the second accommodating chamber, it is difficult for the atomized liquid to impact part of the atomization core assembly 300 in the second accommodating chamber after the atomized liquid is injected by the infusion member 20. Therefore, it is unnecessary to dispose the sealing member in the second accommodating chamber, thereby reducing the production cost of the atomization device 10.

It can be understood that the atomization device in the above embodiments includes, but is not limited to, devices such as aerosol generating device, an aromatherapy machine, a medicine atomizer, a fire protection sprinkler system, a cleaning device, an irrigation device, a cosmetic device, and a laboratory solute extraction device.

The embodiments of the disclosure are introduced above in details. The principles and implementations of the disclosure are described by using specific examples herein, and the descriptions of the foregoing embodiments are merely intended to help understand the method and the core idea of the method of the disclosure. Meanwhile, for those skilled in the art, there will be changes in the specific implementations and application scope based on the ideas of the disclosure. In summary, the content of this specification may not be construed as a limitation of the disclosure.

Apparently, the embodiments described above are only some embodiments of the disclosure, instead of all of them. The accompanying drawings show preferred embodiments of the disclosure, but do not limit the protection scope of the disclosure. The disclosure may be implemented in many different forms. On the contrary, the purpose of providing these embodiments is to make the disclosure of the disclosure be understood more thoroughly and comprehensively. Although the disclosure has been described in detail with reference to the above embodiments, those skilled in the art can still modify the technical solutions described in the above specific embodiments, or equivalently replace some of the technical features. All the equivalent structures, which are made using the contents of the description and the accompanying drawings of the disclosure, and are directly or indirectly used in other related technical fields, still fall within the protection scope of the disclosure.

Although the embodiments of the disclosure have been shown and described, it would be appreciated by those skilled in the art that changes, modifications, combinations, replacements, and variations may be made to these embodiments without departing from the principle and spirit of the disclosure, and the scope of the disclosure is defined by the claims and their equivalents.

What is claimed is:

1. An atomization device, comprising:
an atomization core assembly configured to heat an atomized liquid;
a sealing member; and
a housing defining a first injection hole allowing injection of the atomized liquid, wherein the atomization core assembly and the sealing member are connected to each other and both located within the housing;
wherein the sealing member is at least partially located between the atomization core assembly and the first injection hole.

2. The atomization device according to claim 1, wherein at least part of the sealing member and an outer sidewall of the atomization core assembly cooperatively define an injection gap for flow of the atomized liquid.

3. The atomization device according to claim 2, wherein
the sealing member comprises a limiting portion and a protruding portion connected to the limiting portion, wherein the injection gap is defined by the limiting portion and the outer sidewall of the atomization core assembly; and
the protruding portion is located at one end of the limiting portion close to the first injection hole, and protrudes toward and abuts against the outer sidewall of the atomization core assembly.

4. The atomization device according to claim 3, wherein the atomization device satisfies at least one of:
a width of the injection gap ranges from 0.5 mm to 1 mm;
a thickness of the protruding portion ranges from 1.1 mm to 1.5 mm, a thickness of the limiting portion ranges from 0.5 mm to 1 mm;
the injection gap gradually increases in width in a direction away from the protruding portion; and/or
a height of the first injection hole is equal to a height of the injection gap; or
the thickness of the protruding portion is less than the thickness of the limiting portion to form a communication opening in communication with the injection gap, and the communication opening allows the atomized liquid to flow into the injection gap.

5. The atomization device according to claim 3, wherein
the sealing member further comprises a sealing-member body defining a fixing hole, wherein the atomization core assembly is located in the fixing hole and fixedly connected to the sealing-member body; and
the limiting portion is arranged around an outer periphery of the fixing hole.

6. The atomization device according to claim 5, wherein
the sealing-member body comprises a first sealing-member body and a second sealing-member body connected to the first sealing-member body, wherein the first sealing-member body is close to an inner sidewall of the housing, and the second sealing-member body is close to the atomization core assembly and connected to the sealing member; and
a height of the first sealing-member body is greater than a height of the second sealing-member body.

7. The atomization device according to claim 5, wherein the atomization device satisfies at least one of:
a first elastic protrusion is disposed on an inner sidewall of the fixing hole, wherein the first elastic protrusion abuts against the outer sidewall of the atomization core assembly to enable the atomization core assembly to be in interference fit with the fixing hole; or
a second elastic protrusion is disposed on an outer sidewall of the sealing-member body, wherein the second elastic protrusion abuts against an inner sidewall of the housing to enable the sealing-member body to be in interference fit with the housing.

8. The atomization device according to claim 7, wherein the atomization device satisfies at least one of:
the limiting portion is implemented as a plurality of limiting portions and the protruding portion is implemented as a plurality of protruding portions, wherein the plurality of limiting portions are in one-to-one correspondence with the plurality of protruding portions, and the plurality of limiting portions are symmetrically and circumferentially distributed along the outer periphery of the fixing hole;
the first elastic protrusion is an annular protrusion; or
the first elastic protrusion is implemented as a plurality of first elastic protrusions that are distributed in an axial direction of the fixing hole.

9. The atomization device according to claim 3, wherein the atomization device satisfies at least one of:
the outer sidewall of the atomization core assembly defines a second injection hole, wherein the second injection hole allows the atomized liquid to flow from the injection gap into an interior of the atomization core assembly, an orthographic projection of the limiting portion on the outer sidewall of the atomization core assembly covers at least part of the second injection hole; or
the housing is further provided with an elastic injection plug corresponding to the first injection hole.

10. The atomization device according to claim 3, wherein
a width of the housing is greater than a thickness of the housing, wherein the atomization core assembly and the inner sidewall of the housing cooperatively define two first accommodating chambers that are symmetrically distributed and two second accommodating chambers that are symmetrically distributed; and
the first accommodating chamber is larger than the second accommodating chamber, and both the first injection hole and the limiting portion are located in the first accommodating chamber.

11. The atomization device according to claim 1, wherein the sealing member comprises a blocking portion and a shunting portion, wherein the blocking portion is close to the atomization core assembly and surrounds at least part of a sidewall of the atomization core assembly, and the shunting portion is located on one side of the blocking portion close to the first injection hole and protrudes toward the first injection hole.

12. The atomization device according to claim 11, wherein the shunting portion is located at a middle of the blocking portion to divide the blocking portion into two symmetrical blocking sub-portions.

13. The atomization device according to claim 11, wherein a thickness of the shunting portion is smaller than a diameter of the first injection hole, and an orthographic projection of at least part of the shunting portion on the blocking portion is located within an orthographic projection of the first injection hole on the blocking portion.

14. The atomization device according to claim 11, wherein at least part of the blocking portion and an outer sidewall of the atomization core assembly cooperatively define an injection gap for flow of the atomized liquid.

15. The atomization device according to claim 14, wherein the atomization device satisfies at least one of:
a width of the injection gap ranges from 0.5 mm to 1.2 mm; or
the injection gap gradually increases in width in a direction from a bottom of the atomization core assembly to a top of the atomization core assembly.

16. The atomization device according to claim 11, wherein
the sealing member further comprises a sealing-member body defining a fixing hole, wherein the atomization core assembly located in the fixing hole and fixedly connected to the sealing-member body; and
the blocking portion is arranged around an outer periphery of the fixing hole.

17. The atomization device according to claim 16, wherein
the sealing-member body comprises a first sealing-member body and a second sealing-member body connected to the first sealing-member body, wherein the first sealing-member body is close to an inner sidewall of the housing, and the second sealing-member body is close to the atomization core assembly and connected to the blocking portion; and
a height of the first sealing-member body is greater than a height of the second sealing-member body.

18. The atomization device according to claim 11, wherein the atomization device satisfies at least one of:
the sealing member is implemented as a plurality of sealing members that are symmetrically and circumferentially distributed along an outer periphery of the atomization core assembly; or
the sealing member is elastic.

19. The atomization device according to claim 11, wherein the housing is further provided with an elastic injection plug corresponding to the first injection hole, wherein the elastic injection plug comprises a fixing portion and a flipping portion, wherein the fixing portion is connected to the housing, the flipping portion is connected to the fixing portion, and the flipping portion is detachably connected to the first injection hole and is flippable to block the first injection hole or communicate the first injection hole with an infusion member.

20. The atomization device according to claim 19, wherein
the housing defines a mounting groove and is provided with a limiting portion;
the fixing portion and the flipping portion are located in the mounting groove, the flipping portion has a pressing portion at one end of the flipping portion away from the fixing portion, and the limiting portion is located at one end of an outer periphery of the mounting groove close to the pressing portion; and
when the pressing portion abuts against a sidewall of the limiting portion, the pressing portion and a bottom wall of the mounting groove cooperatively define a deformation chamber, a sidewall of the limiting portion and a sidewall of the mounting groove cooperatively define a limiting chamber, and a sidewall of the elastic injection plug is located within the limiting chamber.
